(19) United States
(12) Patent Application Publication
(10) Pub. No.: US 2017/0065611 A1
(43) Pub. Date: Mar. 9, 2017

(54) TOPICAL FORMULATIONS AND USES THEREOF

(71) Applicant: OCULAR TECHNOLOGIES SARL, Epalinges (CH)

(72) Inventor: Sidney L. WEISS, Randolph, NJ (US)

(73) Assignee: OCULAR TECHNOLOGIES SARL, Epalinges (CH)

(21) Appl. No.: 15/354,568

(22) Filed: Nov. 17, 2016

Related U.S. Application Data
(63) Continuation of application No. PCT/US2015/031788, filed on May 20, 2015.
(60) Provisional application No. 62/002,682, filed on May 23, 2014.

(51) Int. Cl. A61K 31/5575; A61K 47/44; A61K 31/498; A61K 9/08; A61K 31/542; A61K 31/506; A61K 9/00; A61K 47/10

(52) U.S. Cl. CPC A61K 31/5575; A61K 9/0048; A61K 47/44; A61K 47/10; A61K 9/08; A61K 31/542; A61K 31/506; A61K 31/498

(57) ABSTRACT

Provided herein include formulations for topical administration, such as ophthalmic formulations, and methods of using such formulations. In some aspects and embodiments the formulations may include a polyoxyl lipid or fatty acid, and or a polyalkoxylated alcohol and may include nanomicelles. Also include methods of treating or preventing diseases or conditions, such as ocular diseases or conditions.

### CROSS-REFERENCE TO RELATED APPLICATIONS

This application claims priority from International Application No. PCT/US2015/031788, filed May 20, 2015, which claims priority to U.S. application Ser. No. 62/002,682, filed on May 23, 2014, which are hereby incorporated by reference in their entirety.

### FIELD OF THE INVENTION

The present disclosure relates to the field of formulations for topical administration, such as ophthalmic formulations, and methods of using such formulations.

### BACKGROUND OF THE INVENTION

The information provided herein and references cited are provided solely to assist the understanding of the reader, and does not constitute an admission that any of the references or information is prior art to the present invention.

United States Patent Application Nos US2010/0310462 and US2009/0092665 disclose drug delivery systems for ophthalmic use that have nanomicelles that include vitamin E TPGS.

Travoprost involves a formulation for glaucoma or ocular hypertension that includes HCO-40 and a prostaglandin analog as the active ingredient. See dailymed.nlm.nih.gov/dailymed/lookup.cfm?setid=338e7ff4-0d91-4208-a45d-bfa2be52334d on the world-wide web. The active ingredient is present at 0.004%. The formulation includes propylene glycol and does not include nanomicelles. HCO-40 is present in Travoprost at 0.5%. See ema.europa.eu/docs/en_GB/document_library/EPAR_-_Product_Information/human/000665/WC500038389.pcif on the world-wide web.

### SUMMARY OF THE INVENTION

The present disclosure relates to topical formulations such as formulations suitable for ophthalmic administration of an active ingredient such as brinzolamide, latanoprost, brimonidine, bosentan, or mixtures of any two or more thereof (or pharmaceutically acceptable salts, prodrugs or variants thereof). In certain aspects and embodiments, the formulations of the present disclosure may include a polyoxyl lipid or fatty acid, and/or a polyalkoxylated alcohol and may include nanomicelles.

In certain aspects and embodiments as described herein, the formulations as described herein may have certain surprising features and advantages that could not have been predicted prior to the present disclosure. For example, formulations of the instant disclosure may be able to support a dose of an active ingredient such as brinzolamide, latanoprost, brimonidine, bosentan, or mixtures of any two or more thereof (or pharmaceutically acceptable salts, prodrugs or variants thereof) that is surprisingly higher than many prior art formulations and/or which produce surprisingly higher ocular tissue concentrations. The dose of active ingredient or agent used in the formulations described herein may be selected based on various criteria, including the amount that the formulation can support, the desired dose for various therapeutic applications, etc. In this regard, in some embodiments the active agent (such as for ophthalmic administration) may be at least about 0.05 wt %, or at least about 0.08 wt %, or at least about 0.09 wt %, or at least about 0.1 wt %, or at least about 0.15 wt %, or at least about 0.2 wt %, or at least about 0.3 wt %, or at least about 0.4 wt %, or at least about 0.5 wt %, or at least about 0.6 wt %, or at least about 0.7 wt %, or at least about 0.8 wt %, or at least about 0.9 wt %, or at least about 1.0 wt %, or at least about 1.5 wt %, or at least about 2 wt %, or at least about 3 wt %, or at least about 4 wt %, or at least about 5 wt %, or between 0.05 and 5 wt %, or between 0.05 and 0.5 wt %, or between 0.05 and 0.2 wt %, or between 0.08 and 0.12 wt %, or between 0.1 and 0.5 wt %, or between 0.5 and 1 wt %, or between 0.5 and 1.5 wt %, or between 1 and 5 wt %, or between 2 and 4 wt %, or between 4 and 6 wt % of the formulation.

In some embodiments the formulation has nanomicelles with a relatively increased entrapment efficiency; in such embodiments the active agent (such as brinzolamide, latanoprost, brimonidine, or bosentan (or pharmaceutically acceptable salts, prodrugs or variants thereof) for ophthalmic administration) may be at least about 0.05 wt %, or at least about 0.08 wt %, or at least about 0.09 wt %, or at least about 0.1 wt %, or at least about 0.15 wt %; or at least about 0.2 wt %, or at least about 0.3 wt %, or at least about 0.4 wt %, or at least about 0.5 wt %, or at least about 0.6 wt %, or at least about 0.7 wt %, or at least about 0.8 wt %, or at least about 0.9 wt %, or at least about 1.0 wt %, or at least about 1.5 wt %, or at least about 2 wt %, or at least about 3 wt %, or at least about 4 wt %, or at least about 5 wt %, or between 0.05 and 5 wt %, or between 0.05 and 0.5 wt %, or between 0.05 and 0.2 wt %, or between 0.08 and 0.12 wt %, or between 0.1 and 0.5 wt %, or between 0.5 and 1 wt %, or between 0.5 and 1.5 wt %, or between 1 and 5 wt %, or between 2 and 4 wt %, or between 4 and 6 wt % of the formulation and is present in nanomicelles of the formulation.

In certain aspects and embodiments, the formulations of the disclosure are surprisingly effective in dissolving and/or delivering active ingredients (such as brinzolamide, latanoprost, brimonidine, or bosentan (or pharmaceutically acceptable salts, prodrugs or variants thereof)) without a need for organic solvents (such as propylene glycol) that can be an irritant when included in ophthalmic formulations.

In some embodiments, the formulations of the present disclosure are surprisingly stable at high temperatures, for example, temperatures above about 40 degrees C. In some aspects and embodiments the nanomicellular nature of some formulations described herein allow for improved ocular tissue distribution. In certain aspects and embodiments, formulations as described herein are particularly suitable for anterior eye delivery, or posterior eye delivery, or anterior and posterior eye delivery. Moreover, the formulations of certain aspects and embodiments of the disclosure may have the surprising advantage of being adaptable to facilitate delivery of active agents having various sizes or properties; for example, in certain embodiments in formulations that include a polyoxyl castor oil, HCO-60 could be used for active agents having relatively small molecule sizes and HCO-80 and/or HCO-100 could be used for relatively larger sized active agents.

Accordingly, in a first aspect provided is an ophthalmic formulation that includes an active agent such as brinzolamide, latanoprost, brimonidine, bosentan, or mixtures of any two or more thereof (or pharmaceutically acceptable salts, prodrugs or variants thereof), a polyoxyl lipid or fatty acid and a polyalkoxylated alcohol. In some embodiments the formulations includes nanomicelles. In some embodiments the polyoxyl lipid or fatty acid is a polyoxyl castor oil. In some embodiments, the polyoxyl lipid or fatty acid is one or more selected from HCO-40, HCO-60, HCO-80 or HCO-100. In some embodiments the polyoxyl lipid or fatty acid (such as a polyoxyl castor oil such as HCO-40, HCO-60, HCO-80 or HCO-100) is present between 1 and 6%; or 2 and 6%; or 2 and 6%; or 3 and 6%; or 4 and 6%; or 2 and 5%; or 3 and 5%; or 3 and 5%; or 2 and 6%; or about 4%; or greater than 0.7%; or greater than 1%, or greater than 1.5%; or greater than 2%; or greater than 3%; or greater than 4% by weight of the formulation. In some embodiments the polyoxyl lipid is HCO-60. In some embodiments the polyoxyl lipid is HCO-80. In some embodiments the polyoxyl lipid is HCO-100. In some embodiments, the formulation includes a polyalkoxylated alcohol that is octoxynol-40. In some embodiments, the formulation includes a polyalkoxylated alcohol (such as octoxynol-40) present between 0.002 and 4%; or between 0.005 and 3%; or 0.005 and 2%; or 0.005 and 1%; or 0.005 and 0.5%; or 0.005 and 0.1%; or 0.005 and 0.05%; or 0.008 and 0.02%; or about 0.01% by weight of the formulation.

As used herein, the term “polyoxyl lipid or fatty acid” refers to mono- and diesters of lipids or fatty acids and polyoxyethylene diols. Polyoxyl lipids or fatty acids may be numbered (“n”) according to the average polymer length of the oxyethylene units (e.g., 40, 60, 80, 100) as is well understood in the art. The term “n≧40 polyoxyl lipid” means that the ployoxyl lipid or fatty acid has an average oxyethylene polymer length equal to or greater than 40 units. Stearate hydrogenated castor oil and castor oil are common lipids/fatty acids commercially available as polyoxyl lipids or fatty acid, however, it is understood that any lipid or fatty acid could be polyoxylated to become a polyoxyl lipid or fatty acid as contemplated herein. Examples of polyoxyl lipid or fatty acids include without limitation HCO-40, HCO-60, HCO-80, HCO-100, polyoxyl 40 stearate, polyoxyl 35 castor oil, and the like.

In some embodiments of any of the compositions and methods described herein, the average polymer length of the oxyethylene units of a polyoxyl lipid or fatty acid is longer for a relatively larger active ingredient and is shorter for a relatively smaller active ingredient; for example in some embodiments in which the active ingredient is brinzolamide, the polyoxyl lipid is HCO-60 and in some embodiments where the active ingredient is bosentan (which is larger than brinzolamide) the polyoxyl lipid is HCO-80 or HCO-100.

As used herein, the term “micelle” or “nanomicelle” refers to an aggregate (or cluster) of surfactant molecules. Micelles only form when the concentration of surfactant is greater than the critical micelle concentration (CMC). Surfactants are chemicals that are amphipathic, which means that they contain both hydrophobic and hydrophilic groups. Micelles can exist in different shapes, including spherical, cylindrical, and discoidal. A micelle comprising at least two different molecular species is a mixed micelle. In some embodiments, ophthalmic compositions of the present disclosure include an aqueous, clear, mixed micellar solution.

In a second aspect, provided is an ophthalmic formulation, comprising an active agent such as brinzolamide, latanoprost, brimonidine, bosentan, or mixtures of any two or more thereof (or pharmaceutically acceptable salts, prodrugs or variants thereof), and a≧40 polyoxyl lipid or fatty acid. In some embodiments the formulation includes nanomicelles. In some embodiments the polyoxyl lipid or fatty acid is a polyoxyl castor oil. In some embodiments, the polyoxyl lipid or fatty acid is one or more selected from HCO-40, HCO-60, HCO-80 or HCO-100. In some embodiments the polyoxyl lipid or fatty acid (such as a polyoxyl castor oil such as HCO-40, HCO-60, HCO-80 or HCO-100) is present between 0.5 and 2%, or 0.7 and 2%, or 1 and 6%, or 2 and 6%, or 2 and 6%, or 3 and 6%, or 4 and 6%, or 2 and 5%, or 3 and 5%, or 3 and 5%, or 2 and 6%, or about 4%, or greater than 0.7%, or greater than 1%, or greater than 1.5%, or greater than 2%, or greater than 3%, or greater than 4% by weight of the formulation. In some embodiments the polyoxyl lipid is HCO-60. In some embodiments the polyoxyl lipid is HCO-80. In some embodiments the polyoxyl lipid is HCO-100.

In some embodiments, the formulation further includes polyalkoxylated alcohol. In some embodiments, the formulation further includes polyalkoxylated alcohol that is octoxynol-40. In some embodiments, the formulation includes a polyalkoxylated alcohol (such as octoxynol-40) present between 0.002 and 4%, or between 0.005 and 3%, or between 0.005 and 2%, or between 0.005 and 1%, or between 0.005 and 0.5%, or between 0.005 and 0.1%, or between 0.005 and 0.05%, or between 0.008 and 0.02%, or between 0.01 and 0.1%, or between 0.02 and 0.08%, or between 0.005 and 0.08%, or about 0.05%, or about 0.01% by weight of the formulation.

In a third aspect, provided is an ophthalmic formulation, that includes an active ingredient such as brinzolamide, latanoprost, brimonidine, bosentan, or mixtures of any two or more thereof (or pharmaceutically acceptable salts, prodrugs or variants thereof), and a polyoxyl lipid or fatty acid; wherein said polyoxyl lipid or fatty acid is present in an amount equal to or greater than 1 wt % of said formulation. In a similar aspect, provided is an ophthalmic formulation, that includes an active ingredient such as brinzolamide, latanoprost, brimonidine, bosentan, or mixtures of any two or more thereof, and a polyoxyl lipid or fatty acid; wherein said polyoxyl lipid or fatty acid is present in an amount equal to or greater than 0.05 wt % of said formulation. In some embodiments the formulations include nanomicelles. In some embodiments the polyoxyl lipid or fatty acid is a polyoxyl castor oil. In some embodiments, the polyoxyl lipid or fatty acid is one or more selected from HCO-40, HCO-60, HCO-80 or HCO-100. In some embodiments the polyoxyl lipid or fatty acid (such as a polyoxyl castor oil such as HCO-60, HCO-80 or HCO-100) is present between 0.5 and 2%, or 0.7 and 2%, or between 1 and 6%, or 2 and 6%, or 2 and 6%, or 3 and 6%, or 4 and 6%, or 2 and 5%, or 3 and 5%, or 3 and 5%, or 2 and 6%, or about 4%, or greater than 1.5%, or greater than 2%, or greater than 3%, or greater than 4% by weight of the formulation. In some embodiments the polyoxyl lipid is HCO-40. In some embodiments the polyoxyl lipid is HCO-60. In some embodiments the polyoxyl lipid is HCO-80. In some embodiments the polyoxyl lipid is HCO-100.

In some embodiments, the formulation further includes polyalkoxylated alcohol. In some embodiments, the formulation further includes polyalkoxylated alcohol that is octoxynol-40. In some embodiments, the formulation includes a polyalkoxylated alcohol (such as octoxynol-40) present between 0.002 and 4%, or between 0.005 and 3%, or between 0.005 and 2%, or between 0.005 and 1%, or between 0.005 and 0.5%, or between 0.005 and 0.1%, or between 0.005 and 0.05%, or between 0.008 and 0.02%, or between 0.01 and 0.1%, or between 0.02 and 0.08%, or between 0.005 and 0.08%, or about 0.05%, or about 0.01% by weight of the formulation.

In a fourth aspect, provided is an ophthalmic formulation, that includes an active agent such as brinzolamide, latanoprost, brimonidine, bosentan, or mixtures of any two or more thereof (or pharmaceutically acceptable salts, prodrugs or variants thereof), and a polyoxyl lipid or fatty acid; wherein said formulation comprises nanomicelles. In some embodiments the polyoxyl lipid or fatty acid is a polyoxyl castor oil. In some embodiments, the polyoxyl lipid or fatty acid is one or more selected from HCO-40, HCO-60, HCO-80 or HCO-100. In some embodiments the polyoxyl lipid or fatty acid (such as a polyoxyl castor oil such as HCO-40, HCO-60, HCO-80 or HCO-100) is present between 0.5 and 2%, or 0.7 and 2%, or between 1 and 6%, or 2 and 6%, or 2 and 6%, or 3 and 6%, or 4 and 6%, or 2 and 5%, or 3 and 5%, or 3 and 5%, or 2 and 6%, or about 4%, or greater than 0.7%, or greater than 1%, or greater than 1.5%, or greater than 2%, or greater than 3%, or greater than 4% by weight of the formulation. In some embodiments the polyoxyl lipid is HCO-40. In some embodiments the polyoxyl lipid is HCO-60. In some embodiments the polyoxyl lipid is HCO-80. In some embodiments the polyoxyl lipid is HCO-100.

In some embodiments, the formulation further includes polyalkoxylated alcohol. In some embodiments, the formulation further includes polyalkoxylated alcohol that is octoxynol-40. In some embodiments, the formulation includes a polyalkoxylated alcohol (such as octoxynol-40) present between 0.002 and 4%, or between 0.005 and 3%, or between 0.005 and 2%, or between 0.005 and 1%, or between 0.005 and 0.5%, or between 0.005 and 0.1%, or between 0.005 and 0.05%, or between 0.008 and 0.02%, or between 0.01 and 0.1%, or between 0.02 and 0.08%, or between 0.005 and 0.08%, or about 0.05%, or about 0.01% by weight of the formulation.

In a further aspect provided is an ophthalmic formulation, comprising an active agent such as brinzolamide, latanoprost, brimonidine, bosentan, or mixtures of any two or more thereof (or pharmaceutically acceptable salts, prodrugs or variants thereof), 1-5 wt % of one or more selected from the group consisting of HCO-40, HCO-60, HCO-80 and HCO-100; and about 0.002-4 wt % octoxynol-40.

In another aspect, provided is an ophthalmic formulation, comprising an active agent such as brinzolamide, latanoprost, brimonidine, bosentan, or mixtures of any two or more thereof (or pharmaceutically acceptable salts, prodrugs or variants thereof), 1-5 wt % of one or more selected from the group consisting of HCO-40, HCO-60, HCO-80 and HCO-100; and about 0.005-3 wt % octoxynol-40.

In yet another aspect, provided is an ophthalmic formulation, comprising an active agent such as brinzolamide, latanoprost, brimonidine, bosentan, or mixtures of any two or more thereof (or pharmaceutically acceptable salts, prodrugs or variants thereof), 1-5 wt % of one or more selected from the group consisting of HCO-40, HCO-60, HCO-80 and HCO-100; and about 0.005-2 wt % octoxynol-40.

In one aspect, provided is an ophthalmic formulation, comprising an active agent such as brinzolamide, latanoprost, brimonidine, bosentan, or mixtures of any two or more thereof (or pharmaceutically acceptable salts, prodrugs or variants thereof), 1-5 wt % of one or more selected from the group consisting of HCO-40, HCO-60, HCO-80 and HCO-100; and about 0.005-1 wt % octoxynol-40.

In a further aspect provided is an ophthalmic formulation, comprising an active agent such as brinzolamide, latanoprost, brimonidine, bosentan, or mixtures of any two or more thereof (or pharmaceutically acceptable salts, prodrugs or variants thereof), about 4 wt % of HCO-60 and about 0.01 wt % octoxynol-40.

In another aspect provided is an ophthalmic formulation, comprising an active agent such as brinzolamide, latanoprost, brimonidine, bosentan, or mixtures of any two or more thereof (or pharmaceutically acceptable salts, prodrugs or variants thereof), 0.7-1.5 wt % of one or more selected from the group consisting of HCO-40, HCO-60, HCO-80 and HCO-100; and about 0.002-4 wt % octoxynol-40.

In another aspect, provided is an ophthalmic formulation, comprising an active agent such as brinzolamide, latanoprost, brimonidine, bosentan, or mixtures of any two or more thereof (or pharmaceutically acceptable salts, prodrugs or variants thereof), 0.7-1.5 wt % of one or more selected from the group consisting of HCO-40, HCO-60, HCO-80 and HCO-100; and about 0.005-3 wt % octoxynol-40.

In yet another aspect, provided is an ophthalmic formulation, comprising an active agent such as brinzolamide, latanoprost, brimonidine, bosentan, or mixtures of any two or more thereof (or pharmaceutically acceptable salts, prodrugs or variants thereof), 0.7-1.5 wt % of one or more selected from the group consisting of HCO-40, HCO-60, HCO-80 and HCO-100; and about 0.005-2 wt % octoxynol-40.

In one aspect, provided is an ophthalmic formulation, comprising an active agent such as brinzolamide, latanoprost, brimonidine, bosentan, or mixtures of any two or more thereof (or pharmaceutically acceptable salts, prodrugs or variants thereof), 0.7-1.5 wt % of one or more selected from the group consisting of HCO-40, HCO-60, HCO-80 and HCO-100; and about 0.05 wt % octoxynol-40.

In a further aspect provided is an ophthalmic formulation, comprising an active agent such as brinzolamide, latanoprost, brimonidine, bosentan, or mixtures of any two or more thereof (or pharmaceutically acceptable salts, prodrugs or variants thereof), about 1 wt % of HCO-60 and about 0.05 wt % octoxynol-40.

In various embodiments of any of the aspects and embodiments described herein, the formulation includes nanomicelles.

In some embodiments of the aspects and embodiments described herein, the formulation includes a polyoxyl lipid or fatty acid. In some embodiments the polyoxyl lipid or fatty acid is a polyoxyl castor oil. In some embodiments, the polyoxyl lipid or fatty acid is one or more selected from HCO-40, HCO-60, HCO-80 or HCO-100. In some embodiments the polyoxyl lipid or fatty acid (such as a polyoxyl castor oil such as HCO-60, HCO-80 or HCO-100) is present between 0.5 and 2%, or 0.7 and 2%, or 1 and 6%, or 2 and 6%, or 2 and 6%, or 3 and 6%, or 4 and 6%, or 2 and 5%, or 3 and 5%, or 3 and 5%, or 2 and 6%, or about 4%, or greater than 0.7%, or greater than 1%, or greater than 1.5%, or greater than 2%, or greater than 3%, or greater than 4% by weight of the formulation. In some embodiments the polyoxyl lipid is HCO-40. In some embodiments the polyoxyl lipid is HCO-60. In some embodiments the polyoxyl lipid is HCO-80. In some embodiments the polyoxyl lipid is HCO-100.

In some embodiments of the aspects and embodiments disclosed herein, the formulation includes a polyalkoxylated alcohol. In some embodiments, the formulation includes a polyalkoxylated alcohol that is octoxynol-40. In some embodiments, the formulation includes a polyalkoxylated alcohol (such as octoxynol-40) present between 0.002 and 4%, or between 0.005 and 3%, or between 0.005 and 2%, or between 0.005 and 1%, or between 0.005 and 0.5%, or between 0.005 and 0.1%, or between 0.005 and 0.05%, or between 0.008 and 0.02%, or between 0.01 and 0.1%, or between 0.02 and 0.08%, or between 0.005 and 0.08%, or about 0.05%, or about 0.01% by weight of the formulation.

In some embodiments, the active ingredient is a carbonic anhydrase inhibitor such as brinzolamide, methazolamide, dorzolamide, topiramate, punicalin, punicalagin, granatin B, gallagyldilactone, casuarinin, pedunculagin, tellimagrandin I, and the like, as well as pharmaceutically acceptable salts, prodrugs or variants thereof. In some embodiments, the carbonic anhydrase inhibitor is brinzolamide, which has the structure:

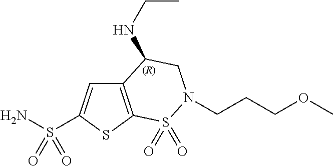

In some embodiments, the active agent is a prostaniod such as latanoprost, having the structure:

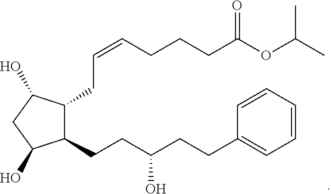 [SMILES: CC(C)OC(=O)CCC/C=C\C[C@@H]1[C@@H](CC[C@@H](O)CCc2ccccc2)[C@@H](O)C[C@@H]1O]

Latanoprost is also known by the brand name of Xalatan manufactured by Pfizer.

In some embodiments, the active agent is an α₂ adrenergic agonist, such as brimonidine, having the structure:

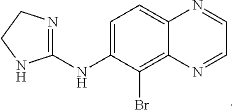 [SMILES: Brc1c(NC2=NCCN2)ccc2nccnc12]

Brimonidine is available as eye drops under the brand names Alphagan and Alphagan-P and as a gel, under the brand name Mirvaso.

In some embodiments, the active ingredient is an endothelin receptor antagonist such as bosentan (that belongs to a class of highly substituted pyrimidine derivatives, with no chiral centers), ambrisentan, sitaxsentan, and the like. In some embodiments, the endothelin receptor antagonist is bosentan, which has the following structure:

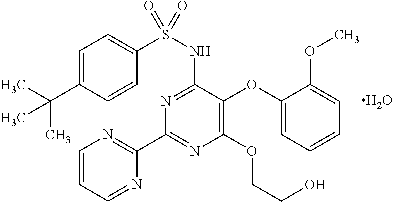

As used herein, the term “pharmaceutically acceptable salts” includes salts of the active compounds that are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

Thus, the compounds contemplated for use in the practice of the present invention may exist as salts, such as with pharmaceutically acceptable acids. The present invention includes such salts. Examples of such salts include hydrochlorides, hydrobromides, sulfates, methanesulfonates, nitrates, maleates, acetates, citrates, fumarates, tartrates (e.g., (+)-tartrates, (−)-tartrates, or mixtures thereof including racemic mixtures), succinates, benzoates, and salts with amino acids such as glutamic acid. These salts may be prepared by methods known to those skilled in the art.

In addition to salt forms, the present disclosure provides compounds which are in a prodrug form. The term “prodrugs” and the like refer, in the usual and customary sense, to compounds that readily undergo chemical changes under physiological conditions to provide the compounds of the present disclosure. Additionally, prodrugs can be converted to the compounds of the present disclosure by chemical or biochemical methods in an ex vivo environment.

As used herein, the terms“pharmaceutically acceptable excipient,” “pharmaceutically acceptable carrier” and the like refer, in the usual and customary sense, to a substance that aids the administration of an active agent to and absorption by a subject and which can be included in the compositions of the present invention without causing a significant adverse toxicological effect on the patient. The term “adverse toxicological effect” and the like refer, in the usual and customary sense, to an indication that a candidate excipient should not be used, as judged by a medical or veterinary practitioner or as known in the art. Unless indicated to the contrary, the terms “drug,” “active agent,” “active ingredient,” “therapeutically active agent,” “therapeutic agent” and like are used synonymously. Non limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, solutions of pharmaceutically acceptable salts (e.g., Ringer's solution), alcohols, oils, gelatins, carbohydrates such as lactose, amylose or starch, fatty acid esters, hydroxymethycellulose, polyvinyl pyrrolidine, polyethylene glycol, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

The instant disclosure further relates to treating or preventing ocular diseases or disorders, for example by local administration of the formulations as described herein.

A patient or subject to be treated by any of the compositions or methods of the present disclosure can mean either a human or a non-human animal. In an embodiment, the present disclosure provides methods for the treatment of an ocular disease in a human patient in need thereof. In an embodiment, the present disclosure provides methods for the treatment of an inflammatory ocular disease in a human patient in need thereof. In another embodiment, the present disclosure provides methods for the treatment of an ocular disease in a veterinary patient in need thereof, including, but not limited to dogs, horses, cats, rabbits, gerbils, hamsters, rodents, birds, aquatic mammals, cattle, pigs, camelids, and other zoological animals.

In some embodiments of the compositions and methods disclosed herein, the active agent comprises a combination of two or more different active ingredients (or pharmaceutically acceptable salts, prodrugs or variants thereof). In some embodiments the active agent comprises a carbonic anhydrase inhibitor (such as brinzolamide) and at least one second active agent such as those described in PCT Publication No. WO 2014/032026 (incorporated by reference herein in its entirety). In some embodiments, the active agent comprises an endothelin receptor antagonist (such as bosentan) and at least one second active agent such as those described in PCT Publication No. WO 2014/032026 (incorporated by reference herein in its entirety).

In some embodiments the active agent includes a carbonic anhydrase inhibitor and an endothelin receptor antagonist. In some embodiments the active agent includes a carbonic anhydrase inhibitor and a resolvin. In some embodiments the active agent includes an endothelin receptor antagonist and a resolvin. In some embodiments the active agent includes a carbonic anhydrase inhibitor and a prostaniod. In some embodiments the active agent includes an endothelin receptor antagonist and a prostaniod. In some embodiments the active agent includes a carbonic anhydrase inhibitor and an α₂ adrenergic agonist. In some embodiments, the active agent includes a an endothelin receptor antagonist and an α₂ adrenergic agonist.

The term “treating” refers to: preventing a disease, disorder or condition from occurring in a cell, a tissue, a system, animal or human which may be predisposed to the disease, disorder and/or condition but has not yet been diagnosed as having it; stabilizing a disease, disorder or condition, i.e., arresting its development; and/or relieving one or more symptoms of the disease, disorder or condition, i.e., causing regression of the disease, disorder and/or condition.

As used herein, a therapeutic that “prevents” a disorder or condition refers to a compound that, in a statistical sample, reduces the occurrence of the disorder or condition in the treated sample relative to an untreated control sample, or delays the onset or reduces the severity of one or more symptoms of the disorder or condition relative to the untreated control sample.

As used herein, the terms “ocular disease,” “ocular condition,” “eye disease,” and “eye condition” refer to diseases/conditions of the eye(s) that can be sight threatening, lead to eye discomfort, and may signal systemic health problems.

As used herein, the term “anterior segment disease” refers to all disorders that affect the eye surface, anterior chamber, iris and ciliary body and lens of the eye. The eye surface is composed of the cornea, conjunctiva, eyelids, lacrimal and meibomian glands, and the interconnecting nerves.

As used herein, the terms “posterior segment eye disease” and “back-of-the-eye disease” refer to all disorders that affect the posterior segment of the eye. A posterior eye disease is a disease which primarily affects a posterior ocular site such as choroid or sclera, vitreous, vitreous chamber, retina, optic nerve, and blood vessels and nerves which vascularize or innervate a posterior ocular site.

Accordingly, in one aspect, provided is a method treating or preventing an ocular disease or condition, that includes locally administering a formulation of any of the aspects or embodiments as disclosed herein. In some embodiments, the ocular disease is an anterior segment disease. In some embodiments, the ocular disease is a posterior segment disease. In some embodiments, the ocular disease is one or more selected from the group consisting of primary open angle glaucoma, primary angle closure glaucoma, ocular hypertension, inflammatory glaucoma, drug-induced glaucoma, diabetic retinopathy (DR), optic neuritis, retrobulbar neuritis, and macular pucker. In one embodiment, the ocular disease is primary open angle glaucoma. In one embodiment, the ocular disease is primary close angle glaucoma. In one embodiment the ocular disease is optic neuritis. In one embodiment the ocular disease is diabetic retinopathy.

### DETAILED DESCRIPTION OF THE PREFERRED EMBODIMENTS

### Active Agents

In accordance various aspects and embodiments of the methods and compositions provided herein, an active agent can be any agent capable of affecting a biological process (e.g., brinzolamide, latanoprost, brimonidine, or bosentan). Active agents (the term active ingredient is used herein interchangably with the term active agent) include drugs, hormones, cytokines, toxins, therapeutic agents, vitamins and the like. In some embodiments an active agent in accordance with the aspects and embodiments disclosed herein is an agent capable of, or approved for, treating or preventing a disease or condition, for example in some embodiments an active agent is capable of, or approved for, treating or preventing an ocular disease or condition.

In some embodiments, the active agent is a carbonic anhydrase inhibitor such as brinzolamide, methazolamide, dorzolamide, topiramate, punicalin, punicalagin, granatin B, gallagyldilactone, casuarinin, pedunculagin, tellimagrandin I, and the like, as well as pharmaceutically acceptable salts, prodrugs or variants thereof.

In some embodiments, the active agent is an endothelin receptor antagonist that belongs to a class of highly substituted pyrimidine derivatives, with no chiral centers. Exemplary endothelin receptor antagonists include bosentan, ambrisentan, sitaxsentan, and the like, as well as pharmaceutically acceptable salts, prodrugs or variants thereof.

In some embodiments, the active agent is a prostanoid such as latanoprost, travoprost, tafluprost, unaprostone, bimatoprost, and the like, as well as pharmaceutically acceptable salts, prodrugs or variants thereof.

In some embodiments, the active agent is an α₂ adrenergic agonist such as apraclonidine, brimonidine, clonidine, detomidine, dexmedetomidine, fadolmidine, guanabenz, guanfacine, lofexidine, medetomidine, methamphetamine, mivazerol, rilmenidine, romifidine, talipexole, tizanidine, tolonidine, xylazine, xylometazoline, and the like, as well as pharmaceutically acceptable salts, prodrugs or variants thereof.

In some embodiments of the compositions and methods disclosed herein, the active agent comprises a combination of two or more different active ingredients. In some embodiments the active agent comprises a carbonic anhydrase inhibitor (such as brinzolamide) and at least one second active agent such as those described in PCT Publication No. WO 2014/032026 (incorporated by reference herein in its entirety). In some embodiments, the active agent comprises an endothelin receptor antagonist (such as bosentan) and at least one second active agent such as those described in PCT Publication No. WO 2014/032026 (incorporated by reference herein in its entirety).

In some embodiments the active agent includes a carbonic anhydrase inhibitor and an endothelin receptor antagonist. In some embodiments the active agent includes a carbonic anhydrase inhibitor and a prostaniod. In some embodiments the active agent includes an endothelin receptor antagonist and a prostanoid. In some embodiments the active agent includes a carbonic anhydrase inhibitor and an α₂ adrenergic agonist. In some embodiments the active agent includes an endothelin receptor antagonist and an α₂ adrenergic agonist. In some embodiments the active agent includes a carbonic anhydrase inhibitor and a beta-adrenergic receptor antagonist. In some embodiments, the active agent includes a an endothelin receptor antagonist and a beta-adrenergic receptor antagonist.

### Ocular Diseases

In various aspects and embodiments the formulations as disclosed herein may be used to treat or prevent an ocular disease or disorder. Ocular diseases and disorders contemplated herein include anterior segment diseases and posterior segment diseases. Exemplary ocular diseases that may in certain embodiments be treated with formulations as disclosed herein include the following.

Open angle glaucoma is a multifactorial optic neuropathy that is chronic and progressive, with a characteristic acquired loss of optic nerve fibers. Such loss develops in the presence of open anterior chamber angles, characteristic visual field abnormalities, and intraocular pressure that is too high for the continued health of the eye.

Closed angle glaucoma is caused by a rapid or sudden increase in intraocular pressure.

Diabetes can affect the eye in a number of ways. Diabetic retinopathy (DR) is a complication of diabetes that results from damage to the blood vessels of the light-sensitive tissue at the back of the eye (the retina). At first, diabetic retinopathy may cause no symptoms or only mild vision problems. Eventually, however, diabetic retinopathy can result in blindness. Diabetic macular edema (DME) is the swelling of the retina in diabetes mellitus due to leaking of fluid from blood vessels within the macula.

Proliferative vitreoretinopathy (PVR) is scar tissue formation within the eye. “Proliferative” because cells proliferate and “vitreoretinopathy” because the problems involve the vitreous and retina. In PVR scar tissue forms in sheets on the retina which contract. This marked contraction pulls the retina toward the center of the eye and detaches and distorts the retina severely. PVR can occur both posteriorly and anteriorly with folding of the retina both anteriorly and circumferentially.

Optic neuritis occurs when the optic nerve becomes inflamed and the myelin sheath becomes damaged or is destroyed. Nerve damage that occurs in the section of the optic nerve located behind the eye, is called retrobulbar neuritis, which is another term sometimes used for optic neuritis.

Also known as macular pucker, epiretinal membrane is a scar-tissue like membrane that forms over the macula. It typically progresses slowly and affects central vision by causing blurring and distortion. As it progresses, the pulling of the membrane on the macula may cause swelling.

### Additional Formulation Ingredients

The compositions of the present disclosure may also contain other components such as, but not limited to, additives, adjuvants, buffers, tonicity agents, bioadhesive polymers, and preservatives. In any of the compositions of this disclosure for topical to the eye, the mixtures are preferably formulated at about pH 5 to about pH 8. This pH range may be achieved by the addition of buffers to the composition as described in the examples. In an embodiment, the pH range in the composition in a formulation is about pH 6.6 to about pH 7.0. It should be appreciated that the compositions of the present disclosure may be buffered by any common buffer system such as phosphate, borate, acetate, citrate, carbonate and borate-polyol complexes, with the pH and osmolality adjusted in accordance with well-known techniques to proper physiological values. The mixed micellar compositions of the present disclosure are stable in buffered aqueous solution. That is, there is no adverse interaction between the buffer and any other component that would cause the compositions to be unstable.

Tonicity agents include, for example, mannitol, sodium chloride, xylitol, etc. These tonicity agents may be used to adjust the osmolality of the compositions. In one aspect, the osmolality of the formulation is adjusted to be in the range of about 250 to about 350 mOsmol/kg. In a preferred aspect, the osmolality of the formulation is adjusted to between about 280 to about 300 mOsmol/kg.

An additive such as a sugar, a glycerol, and other sugar alcohols, can be included in the compositions of the present disclosure. Pharmaceutical additives can be added to increase the efficacy or potency of other ingredients in the composition. For example, a pharmaceutical additive can be added to a composition of the present disclosure to improve the stability of the calcineurin inhibitor or mTOR inhibitor, to adjust the osmolality of the composition, to adjust the viscosity of the composition, or for another reason, such as effecting drug delivery. Non-limiting examples of pharmaceutical additives of the present disclosure include sugars, such as, trehalose, mannose, D-galactose, and lactose. In an embodiment, the sugars can be incorporated into a composition prior to hydrating the thin film (i.e., internally). In another embodiment, the sugars can be incorporated into a composition during the hydration step (i.e., externally) (see Example 17). In an embodiment, an aqueous, clear, mixed micellar solution of the present disclosure includes additives such as sugars.

In an embodiment, compositions of the present disclosure further comprise one or more bioadhesive polymers. Bioadhesion refers to the ability of certain synthetic and biological macromolecules and hydrocolloids to adhere to biological tissues. Bioadhesion is a complex phenomenon, depending in part upon the properties of polymers, biological tissue, and the surrounding environment. Several factors have been found to contribute to a polymer's bioadhesive capacity: the presence of functional groups able to form hydrogen bridges (—OH, COOH), the presence and strength of anionic charges, sufficient elasticity for the polymeric chains to interpenetrate the mucous layer, and high molecular weight. Bioadhesion systems have been used in dentistry, orthopedics, ophthalmology, and in surgical applications. However, there has recently emerged significant interest in the use of bioadhesive materials in other areas such as soft tissue-based artificial replacements, and controlled release systems for local release of bioactive agents. Such applications include systems for release of drugs in the buccal or nasal cavity, and for intestinal or rectal administration.

In an embodiment, a composition of the present disclosure includes at least one bioadhesive polymer. The bioadhesive polymer can enhance the viscosity of the composition and thereby increase residence time in the eye. Bioadhesive polymers of the present disclosure include, for example, carboxylic polymers like Carbopol® (carbomers), Noveon® (polycarbophils), cellulose derivatives including alkyl and hydroxyalkyl cellulose like methylcellulose, hydroxypropylcellulose, carboxymethylcellulose, gums like locust beam, xanthan, agarose, karaya, guar, and other polymers including but not limited to polyvinyl alcohol, polyvinyl pyrollidone, polyethylene glycol, Pluronic® (Poloxamers), tragacanth, and hyaluronic acid; phase-transition polymers for providing sustained and controlled delivery of enclosed medicaments to the eye (e.g., alginic acid, carrageenans (e.g., Eucheuma), xanthan and locust bean gum mixtures, pectins, cellulose acetate phthalate, alkylhydroxyalkyl cellulose and derivatives thereof, hydroxyalkylated polyacrylic acids and derivatives thereof, poloxamers and their derivatives, etc. Physical characteristics in these polymers can be mediated by changes in environmental factors such as ionic strength, pH, or temperature alone or in combination with other factors. In an embodiment, the optional one or more bioadhesive polymers is present in the composition from about 0.01 wt % to about 10 wt %/volume, preferably from about 0.1 to about 5 wt %/volume. In an embodiment, the compositions of the present disclosure further comprise at least one hydrophilic polymer excipient selected from, for example, PVP-K-30, PVP-K-90, HPMC, HEC, and polycarbophil. In an embodiment, the polymer excipient is selected from PVP-K-90, PVP-K-30 or HPMC. In an embodiment, the polymer excipient is selected from PVP-K-90 or PVP-K-30.

In an embodiment, if a preservative is desired, the compositions may optionally be preserved with any of many well-known preservatives, including benzyl alcohol with/without EDTA, benzalkonium chloride, chlorhexidine, Cosmocil® CQ, or Dowicil® 200. In certain embodiments, it may be desirable for a formulation as described herein to not include any preservatives. In this regard, preservatives may in some embodiments not be necessary or desirable in formulations included in single use containers. In other embodiments it may be advantageous to include preservatives, such as in certain embodiments in which the formulations are included in a multiuse container.

The ophthalmic compositions can be administered topically to the eye as biocompatible, aqueous, clear mixed micellar solutions. The compositions have the drugs incorporated and/or encapsulated in micelles which are dispersed in an aqueous medium.

### Non-Limiting List of Exemplary Embodiments

In addition to the aspects and embodiments described and provided elsewhere in this disclosure, the following non-limiting list of particular embodiments are specifically contemplated.
1. An ophthalmic formulation, comprising an active agent selected from the group consisting of brinzolamide, latanoprost, brimonidine, and bosentan (or pharmaceutically acceptable salts, prodrugs or variants thereof), a polyoxyl lipid or fatty acid and a polyalkoxylated alcohol.
2. An ophthalmic formulation, comprising an active agent selected from the group consisting of brinzolamide, latanoprost, brimonidine, and bosentan (or pharmaceutically acceptable salts, prodrugs or variants thereof), and a n≧40 polyoxyl lipid or fatty acid.
3. An ophthalmic formulation, comprising an active ingredient selected from the group consisting of brinzolamide, latanoprost, brimonidine, and bosentan (or pharmaceutically acceptable salts, prodrugs or variants thereof) and a polyoxyl lipid or fatty acid; wherein said polyoxyl lipid or fatty acid is present in an amount equal to or greater than 1 wt % of said formulation.
4. An ophthalmic formulation, comprising an active agent selected from the group consisting of brinzolamide, latanoprost, brimonidine, and bosentan (or pharmaceutically acceptable salts, prodrugs or variants thereof), and a polyoxyl lipid or fatty acid; wherein said formulation comprises nanomicelles.
5. An ophthalmic formulation, comprising an active agent selected from the group consisting of brinzolamide, latanoprost, brimonidine, and bosentan (or pharmaceutically acceptable salts, prodrugs or variants thereof), 1-5 wt % of one or more selected from the group consisting of HCO-40, HCO-60, HCO-80 and HCO-100; and about 0.01-0.1 wt % octoxynol-40.
6. An ophthalmic formulation, comprising greater than 0.2 wt % of an active agent selected from the group consisting of brinzolamide, latanoprost, brimonidine, and bosentan (or pharmaceutically acceptable salts, prodrugs or variants thereof), 1-5 wt % of one or more selected from the group consisting of HCO-40, HCO-60, HCO-80 and HCO-100; and about 0.01-0.1 wt % octoxynol-40.
7. An ophthalmic formulation, comprising greater than 0.5 wt % of an active agent selected from the group consisting of brinzolamide, latanoprost, brimonidine, and bosentan (or pharmaceutically acceptable salts, prodrugs or variants thereof), 1-5 wt % of one or more selected from the group consisting of HCO-40, HCO-60, HCO-80 and HCO-100; and about 0.01-0.1 wt % octoxynol-40.
8. An ophthalmic formulation, comprising an active agent selected from the group consisting of brinzolamide, latanoprost, brimonidine, and bosentan (or pharmaceutically acceptable salts, prodrugs or variants thereof), 1.5-4 wt % of one or more polyoxyl lipids selected from the group consisting of HCO-40, HCO-60, HCO-80 and HCO-100; and about 0.01-0.1 wt % octoxynol-40.
9. An ophthalmic formulation, comprising greater than 0.2 wt % of an active agent selected from the group consisting of brinzolamide, latanoprost, brimonidine, and bosentan (or pharmaceutically acceptable salts, prodrugs or variants thereof), 1.5-4 wt % of polyoxyl lipids or fatty acids; and about 0.01-0.1 wt % octoxynol-40.
10. An ophthalmic formulation, comprising an active agent selected from the group consisting of brinzolamide, latanoprost, brimonidine, and bosentan (or pharmaceutically acceptable salts, prodrugs or variants thereof), 1.5-4 wt % of polyoxyl lipids or fatty acids; and about 0.01-0.1 wt % octoxynol-40; wherein the formulation comprises nanomicelles.
11. An ophthalmic formulation, comprising greater than 0.2 wt % of a hydrophobic active agent selected from the group consisting of brinzolamide, latanoprost, brimonidine, and bosentan (or pharmaceutically acceptable salts, prodrugs or variants thereof), 1.5-4 wt % of polyoxyl lipids or fatty acids; and about 0.01-0.1 wt % octoxynol-40; wherein the formulation comprises nanomicelles.
12. An ophthalmic formulation, comprising an active agent selected from the group consisting of brinzolamide, latanoprost, brimonidine, and bosentan (or pharmaceutically acceptable salts, prodrugs or variants thereof), about 4 wt % of one or more selected from the group consisting of HCO-40, HCO-60, HCO-80 and HCO-100; and about 0.01-0.1 wt % octoxynol-40.
13. An ophthalmic formulation, comprising an active agent selected from the group consisting of brinzolamide, latanoprost, brimonidine, and bosentan (or pharmaceutically acceptable salts, prodrugs or variants thereof), about 4 wt % of HCO-60 and about 0.01-0.1 wt % octoxynol-40.
14. An ophthalmic formulation, comprising an active agent selected from the group consisting of brinzolamide, latanoprost, brimonidine, and bosentan (or pharmaceutically acceptable salts, prodrugs or variants thereof), 1-5 wt % of one or more selected from the group consisting of HCO-40, HCO-60, HCO-80 and HCO-100; and about 0.01 wt % octoxynol-40.
15. An ophthalmic formulation, comprising greater than 0.2 wt % of an active agent selected from the group consisting of brinzolamide, latanoprost, brimonidine, and bosentan (or pharmaceutically acceptable salts, prodrugs or variants thereof), 1-5 wt % of one or more selected from the group consisting of HCO-40, HCO-60, HCO-80 and HCO-100; and about 0.01 wt % octoxynol-40.
16. An ophthalmic formulation, comprising greater than 0.5 wt % of an active agent selected from the group consisting of brinzolamide, latanoprost, brimonidine, and bosentan (or pharmaceutically acceptable salts, prodrugs or variants thereof), 1-5 wt % of one or more selected from the group consisting of HCO-40, HCO-60, HCO-80 and HCO-100; and about 0.01 wt % octoxynol-40.
17. An ophthalmic formulation, comprising an active agent selected from the group consisting of brinzolamide, latanoprost, brimonidine, and bosentan (or pharmaceutically acceptable salts, prodrugs or variants thereof), 1.5-4 wt % of one or more polyoxyl lipids selected from the group consisting of HCO-40, HCO-60, HCO-80 and HCO-100; and about 0.01 wt % octoxynol-40.
18. An ophthalmic formulation, comprising greater than 0.2 wt % of an active agent selected from the group consisting of brinzolamide, latanoprost, brimonidine, and bosentan (or pharmaceutically acceptable salts, prodrugs or variants thereof), 1.5-4 wt % of polyoxyl lipids or fatty acids; and about 0.01 wt % octoxynol-40.
19. An ophthalmic formulation, comprising an active agent selected from the group consisting of brinzolamide, latanoprost, brimonidine, and bosentan (or pharmaceutically acceptable salts, prodrugs or variants thereof), 1.5-4 wt % of polyoxyl lipids or fatty acids; and about 0.01 wt % octoxynol-40; wherein the formulation comprises nanomicelles.
20. An ophthalmic formulation, comprising a hydrophobic active agent selected from the group consisting of brinzolamide, latanoprost, brimonidine, and bosentan (or pharmaceutically acceptable salts, prodrugs or variants thereof), 1.5-4 wt % of polyoxyl lipids or fatty acids; and about 0.01 wt % octoxynol-40; wherein the formulation comprises nanomicelles.
21. An ophthalmic formulation, comprising an active agent selected from the group consisting of brinzolamide, latanoprost, brimonidine, and bosentan (or pharmaceutically acceptable salts, prodrugs or variants thereof), about 4 wt % of one or more selected from the group consisting of HCO-40, HCO-60, HCO-80 and HCO-100; and about 0.01 wt % octoxynol-40.
22. An ophthalmic formulation, comprising greater than 0.2 wt % of an active agent selected from the group consisting of brinzolamide, latanoprost, brimonidine, and bosentan (or pharmaceutically acceptable salts, prodrugs or variants thereof), about 4 wt % of HCO-60 and about 0.01 wt % octoxynol-40.
23. The formulation of any of the preceding embodiments, wherein said polyoxyl lipid or fatty acid is between 0.5 and 6% by weight of said formulation.
24. The formulation of any of the preceding embodiments, wherein said polyoxyl lipid or fatty acid is between 0.5 and 2% by weight of said formulation.
25. The formulation of any of the preceding embodiments, wherein said polyoxyl lipid or fatty acid is between 0.5 and 3% by weight of said formulation.
26. The formulation of any of the preceding embodiments, wherein said polyoxyl lipid or fatty acid is between 0.5 and 4% by weight of said formulation.
27. The formulation of any of the preceding embodiments, wherein said polyoxyl lipid or fatty acid is between 0.5 and 5% by weight of said formulation.
28. The formulation of any of the preceding embodiments, wherein said polyoxyl lipid or fatty acid is between 1 and 6% by weight of said formulation.
29. The formulation of any of the preceding embodiments, wherein said polyoxyl lipid or fatty acid is between 1 and 2% by weight of said formulation.
30. The formulation of any of the preceding embodiments, wherein said polyoxyl lipid or fatty acid is between 1 and 3% by weight of said formulation.
31. The formulation of any of the preceding embodiments, wherein said polyoxyl lipid or fatty acid is between 1 and 4% by weight of said formulation.
32. The formulation of any of the preceding embodiments, wherein said polyoxyl lipid or fatty acid is between 1 and 5% by weight of said formulation.
33. The formulation of any of the preceding embodiments, wherein said polyoxyl lipid or fatty acid is between 1 and 6% by weight of said formulation.
34. The formulation of any of the preceding embodiments, wherein said polyoxyl lipid or fatty acid is between 2 and 6% by weight of said formulation.
35. The formulation of any of the preceding embodiments, wherein said polyoxyl lipid or fatty acid is between 3 and 6% by weight of said formulation.
36. The formulation of any of the preceding embodiments, wherein said polyoxyl lipid or fatty acid is between 4 and 6% by weight of said formulation.
37. The formulation of any of the preceding embodiments, wherein said polyoxyl lipid or fatty acid is between 2 and 5% by weight of said formulation.
38. The formulation of any of the preceding embodiments, wherein said polyoxyl lipid or fatty acid is between 3 and 5% by weight of said formulation.
39. The formulation of any of the preceding embodiments, wherein said polyoxyl lipid or fatty acid is about 4% by weight of said formulation.
40. The formulation of any of the preceding embodiments, wherein said polyoxyl lipid or fatty acid is greater than about 0.7% by weight of said formulation.
41. The formulation of any of the preceding embodiments, wherein said polyoxyl lipid or fatty acid is greater than about 1% by weight of said formulation.
42. The formulation of any of the preceding embodiments, wherein said polyoxyl lipid or fatty acid is greater than about 1.5% by weight of said formulation.
43. The formulation of any of the preceding embodiments, wherein said polyoxyl lipid or fatty acid is greater than about 2% by weight of said formulation.
44. The formulation of any of the preceding embodiments, wherein said polyoxyl lipid or fatty acid is greater than about 3% by weight of said formulation.
45. The formulation of any of the preceding embodiments, wherein said polyalkoxylated alcohol if present is between 0.002 and 4% by weight of said formulation.
46. The formulation of any of the preceding embodiments, wherein said polyalkoxylated alcohol if present is between 0.005 and 3% by weight of said formulation.
47. The formulation of any of the preceding embodiments, wherein said polyalkoxylated alcohol if present is between 0.005 and 2% by weight of said formulation.
48. The formulation of any of the preceding embodiments, wherein said polyalkoxylated alcohol if present is between 0.005 and 1% by weight of said formulation.
49. The formulation of any of the preceding embodiments, wherein said polyalkoxylated alcohol if present is between 0.005 and 0.5% by weight of said formulation.
50. The formulation of any of the preceding embodiments, wherein said polyalkoxylated alcohol if present is between 0.005 and 0.1% by weight of said formulation.
51. The formulation of any of the preceding embodiments, wherein said polyalkoxylated alcohol if present is between 0.005 and 0.05% by weight of said formulation.
52. The formulation of any of the preceding embodiments, wherein said polyalkoxylated alcohol if present is between 0.008 and 0.02% by weight of said formulation.
53. The formulation of any of the preceding embodiments, wherein said polyalkoxylated alcohol if present is about 0.01% by weight of said formulation.
54. The formulation of any of the preceding embodiments, wherein said active agent is present in said formulation in an amount greater than 0.2% by weight.
55. The formulation of any of the preceding embodiments, wherein said active agent is present in said formulation in an amount greater than 0.3% by weight.
56. The formulation of any of the preceding embodiments, wherein said active agent is present in said formulation in an amount greater than 0.4% by weight.
57. The formulation of any of the preceding embodiments, wherein said active agent is present in said formulation in an amount greater than 0.5% by weight.
58. The formulation of any of the preceding embodiments, wherein said active agent is present in said formulation in an amount greater than 0.6% by weight.
59. The formulation of any of the preceding embodiments, wherein said active agent is present in said formulation in an amount greater than 0.7% by weight.
60. The formulation of any of the preceding embodiments, wherein said active agent is present in said formulation in an amount greater than 0.8% by weight.
61. The formulation of any of the preceding embodiments, wherein said active agent is present in said formulation in an amount greater than 0.9% by weight.
62. The formulation of any of the preceding embodiments, wherein said active agent is present in said formulation in an amount greater than 1% by weight.
63. The formulation of any of the preceding embodiments, wherein said active agent is present in said formulation in an amount greater than 1.5% by weight.
64. The formulation of any of the preceding embodiments, wherein said active agent is present in said formulation in an amount greater than 2% by weight.
65. The formulation of any of the preceding embodiments, wherein said active agent is present in said formulation in an amount greater than 3% by weight.
66. The formulation of any of the preceding embodiments, wherein said active agent is present in said formulation in an amount greater than 4% by weight.
67. The formulation of any of the preceding embodiments, wherein said polyoxyl lipid or fatty acid is a polyoxyl castor oil.
68. The formulation of any of the preceding embodiments, wherein said polyoxyl lipid or fatty acid is one or more selected from HCO-60, HCO-80 or HCO-100.
69. The formulation of any of the preceding embodiments, wherein said polyoxyl lipid or fatty acid is HCO-60.
70. The formulation of any of the preceding embodiments, wherein said active agent is brinzolamide, or a pharmaceutically acceptable salt thereof
71. The formulation of any of the preceding embodiments, wherein said active agent is bosentan, or a pharmaceutically acceptable salt thereof
72. The formulation of any of the preceding embodiments, wherein said active agent comprises a combination of two different agents.
73. The formulation of any of the preceding embodiments, wherein the active agent comprises at least one of a carbonic anhydrase inhibitor and/or an endothelin receptor antagonist that belongs to a class of highly substituted pyrimidine derivatives, with no chiral centers, and at least one additional active agent selected from the group consisting of a prostanoid, an α₂ adrenergic agonist, a resolvin or resolvin-like compound, a steroid (such as a corticosteroid), cyclosporine A, and voclosporin.
74. The formulation of any of the preceding embodiments, wherein the active agent further comprises a prostanoid and/or an α₂ adrenergic agonist.
75. The formulation of any of the preceding embodiments, wherein the active agent comprises a carbonic anhydrase inhibitor.
76. The formulation of any of the preceding embodiments, wherein the active agent comprises an endothelin receptor antagonist that belongs to a class of highly substituted pyrimidine derivatives, with no chiral centers.
77. The formulation of any of the preceding embodiments, wherein the active agent further comprises a prostanoid, an α₂ adrenergic agonist, and/or a corticosteroid.
78. The formulation of any of the preceding embodiments, wherein said formulation does not include preservatives.
79. The formulation of any of the preceding embodiments, wherein said formulation does not include benzyl alcohol with/without EDTA, benzalkonium chloride, chlorhexidine, Cosmocil® CQ, or Dowicil® 200.
80. A method of treating or preventing an ocular disease or condition, said method comprising topically administering a formulation of any of the preceding embodiments.
81. A method of treating or preventing an ocular disease or condition, said method comprising topically administering a formulation of any of the preceding embodiments; wherein said disease is an anterior segment disease.
82. A method of treating or preventing an ocular disease or condition, said method comprising topically administering a formulation of any of the preceding embodiments; wherein said disease is an posterior segment disease.
83. A method of treating or preventing an ocular disease or condition, said method comprising topically administering a formulation of any of the preceding embodiments; wherein said disease is one or more selected from the group consisting of open angle glaucoma, closed angle glaucoma, diabetic retinopathy (DR), diabetic macular edema (DME), optic neuritis and retrobulbar neuritis.
84. A method of treating or preventing an ocular disease or condition, said method comprising topically administering a formulation of any of the preceding embodiments; wherein said disease is dry eye syndrome.

The following examples are provided to further illustrate aspects of the invention. These examples are non-limiting and should not be construed as limiting any aspect of the invention.

### Example 1

### Preparation of Mixed Nanomicellar Formulations Using Dialysis Method

Mixed nanomicellar formulations of brinzolamide, latanoprost, brimonidine, and bosentan are prepared by dialysis method with varying ratios of polymers and the drug. Experimental design software, JMP 9.0 is used to design the experiments and analyze the results. Accurately weighted quantities of two polymers (i.e., polyoxyl hydrogenated castor-60 (HCO-60) and octoxynol-40 (Oc-40)) are dissolved in 300 microliter volume of propylene glycol. Eighty microliter of propylene glycol containing brinzolamide or bosentan in PG, or 80 mg of brinzolamide or bosentan is added to this polymer mixture and vortex mixed to obtain a clear homogenous solution. The volume of the mixture is made up (500 microliters) with propylene glycol. The solution is vortex mixed to obtain a homogenous solution. A volume of 500 microliter distilled deionized water is added to this mixture to obtain a total volume of 1000 microliter (1 milliliter). Addition of water to the drug-polymer mixture in organic solvent should spontaneously generate micelles thereby entrapping the pharmaceutical active agent in the hydrophobic core of mixed nanomicelles.

The mixture is transferred to a dialysis bag (molecular weight cut off 1000) and transferred to a beaker containing one liter of distilled deionized water. The beaker and its contents are protected from sunlight by covering with aluminum foil and are kept under slow constant stirring at room temperature. Dialysis of the mixture is carried over a period of 24 h to remove the water soluble organic solvent, propylene glycol, from the mixture. Water in the dialysis chamber is changed at predetermined time points: 1 h, 2 h, 4 h, 6 h, 12 h and 24 h. At the end of dialysis (24 h), the contents of the dialysis bag are carefully transferred to a 15-mL centrifuge tube and the formulations are subjected to sonication in a water bath (time range from 0 min to 5 min). The final volume is made up with 2× phosphate buffered saline and the pH of the formulation adjusted to 6.5±0.1. The resultant formulation is filtered with a 0.22 micrometer nylon filter to remove any foreign particulate matter.

The prepared formulations are subjected to various tests such as entrapment efficiency, loading efficiency, mixed nanomicellar size and polydispersity index.

Mixed Nanomicellar Size and Polydispersity Index:

The formulation size and polydispersity index are determined with Zetasizer, Malvern Instruments, NJ. In brief, approximately 1 ml of each formulation is transferred to a cuvette and placed in the instrument. A laser beam of light is used to determine the mixed nanomicellar size.

Entrapment Efficiency:

To determine the entrapment efficiency of the formulation, all the prepared formulations are subjected to an entrapment efficiency test. Briefly, formulations are vortex mixed for homogeneity and 1 mL is transferred to a fresh (1.5 mL) eppendorf tube. Each formulation is lyophilized to obtain a solid at the bottom of eppendorf tube. The obtained solid is suspended in 1 mL of organic solvent (diethyl ether) to generate reverse micelles and release the drug into the external organic solvent. The organic solvent is evaporated overnight in speed vacuum. The resultant reversed micelles are resuspended in 1 mL of 2-propanol (dilution factor is taken into account) and further diluted to determine the concentration of brinzolamide and bosentan entrapped in each micellar preparation with HPLC. The entrapment efficiency of the formulation is calculated with the following formula (wherein MNF=Mixed Nanomicellar Formulation):

$$Entrapment efficiency = \frac{( amount of drug quantified in MNF )}{Amount of drug added in the MNF} \times 100$$

Drug Quantification by an HPLC Method:

In vitro analysis of brinzolamide, latanoprost, brimonidine, and bosentan is performed by a reversed phase high performance liquid chromatography (RP-HPLC) method with a Shimadzu HPLC pump (Shimadzu, Shimadzu Scientific instruments, Columbia, Md.), Alcott autosampler (model 718 AL), Shimadzu UV/Visible detector (Shimadzu, SPD-20A/20AV, USA), ODS column (5 μm, 150×4.6 mm) thermostated at 40°±1 C and Hewlett Packard HPLC integrator (Hewlett Packard, Palo Alto, Calif.). The mobile phase is comprised of methanol (MeOH), water and trifluoroacetic acid (TFA) (70:30:0.05% v/v) which is set at a flow rate of 0.5 mL/min. Detection wavelength is set at 272 nm. The sample tray temperature is maintained at 4° C. Calibration curve (0.5 to 5 μg/mL) for brinzolamide and bosentan is prepared by making appropriate dilutions from the stock solution in 2-propanol. An injection volume of 10 μl is injected into the HPLC column for analysis. All the standards and samples prepared are stored at 4° C. before and during the analysis.

### Example 2

### Preparation of Mixed Nanomicellar Formulation Using Ethyl Acetate Solvent Evaporation Method

Mixed nanomicellar formulation encapsulating brinzolamide, latanoprost, brimonidine, and/or bosentan is prepared by solvent evaporation method in two steps: 1) Preparation of basic formulation and 2) rehydration. In step one, brinzolamide, latanoprost, brimonidine, or bosentan, HCO-60 and octoxynol-40 are dissolved separately in 0.3 mL of ethyl acetate. These three solutions are mixed together in 15-mL centrifuge tube. The resultant mixture is vortexed to obtain a homogenous solution. Ethyl acetate solvent is removed with speed vacuum to obtain a solid thin film. The residue is kept overnight under high vacuum at room temperature to remove residual organic solvent.

In step two, the resultant thin film is hydrated with 1 mL of double distilled deionized water by vortexing the solution. The rehydrated formulation is suspended in 2× phosphate buffer solution, (pH 6.5). It is filtered through 0.2 μm nylon filter membrane to remove the unentrapped drug aggregates and other foreign particulates. The entrapment of brinzolamide and/or bosentan is determined by RP-HPLC following disruption of the micelles and solubilization of the brinzolamide and/or bosentan in the diluent (2-propanol) as described below.

The prepared formulations are subjected to various tests such as entrapment efficiency, loading efficiency, mixed nanomicellar size and polydispersity index according to the methods described in Example 1.

Weight percent of drug loaded into MNF is determined following the method for entrapment efficiency. Size and polydispersity index of the formulations is determined with Malvern zetasizer as described above. The formulations appear clear and have small size and narrow size distribution.

### Example 3

### Preparation of Mixed Nanomicellar Formulation Using Melt Method

Two hundred milligrams of hydrogenated castor oil-60 (HCO-60) (4 wt %) is weighed and transferred to a 10 mL round bottom flask (RBF). The neck of the round bottom flask is closed with aluminum foil, sealed with parafilm and transferred to a water bath set at 40° C. The round bottom flask is left overnight in the water bath to liquefy/melt the HCO-60. On the next day, ten microliters of octoxynol-40 is diluted 100 fold and allowed to equilibrate at 40° C. for 1 h in a water bath. Similarly, brinzolamide, latanoprost, brimonidine, and/or bosentan are allowed to equilibrate at 40° C. in the water bath for 1 h. To the HCO-60 melt, 50 μL of 100 fold diluted octoxynol-40 (0.01 wt %) are added at 40° C. To the above mixture, ˜20 μL of brinzolamide and/or bosentan at 40° C. are added and stirred. To this mixture distilled deionized water, approx. 2 mL, equilibrated at 40° C. is slowly added and stirred. The neck of the round bottom flask is closed with aluminum foil and sealed with parafilm. The solution is stirred in a water bath set at 40° C. overnight protected from light (covering with aluminum foil). On the next day, the above obtained solution at 40° C. is removed from the water bath and allowed to cool to room temperature and observed for clarity. Two milliliters of phosphate buffer (2×) is added to the above prepared solution (phosphate buffer is previously prepared and the pH is adjusted to 5.5). The volume of the formulation is made up to 5 mL with the 2× phosphate buffer saline. The prepared formulation is filtered with 0.2 μm nylon filter and stored at 4° C.

The prepared formulations are subjected to various tests such as entrapment efficiency, loading efficiency, mixed nanomicellar size and polydispersity index according to the methods described in Example 1.

### Example 4

### Preparation of Mixed Nanomicellar Formulation Using Second Melt Method

The preparation of MNF encapsulating brinzolamide, latanoprost, brimonidine, and/or bosentan can be divided into two steps. As an example, the development of 3.0 wt % HCO-40 or HCO-60 MNF encapsulating 0.4 wt % brinzolamide and/or bosentan is described below. In step 1, HCO-40 or HCO-60, 150 mg, is thermostated at 40° C. in a water bath to melt and result in a clear thick viscous liquid. To this melt polymer, brinzolamide, latanoprost, brimonidine, and/or bosentan (˜20 mg), thermostated at 40° C., is added and mixed for homogenous distribution. The mixture is allowed to reach room temperature, resulting in a pale yellow color viscous liquid with HCO-40 and waxy solid with HCO-60. Further, to solidify the viscous liquid of HCO-40, the mixture is stored at 4° C. (in refrigerator).

In step 2, the pellet and/or viscous liquid is allowed to reach room temperature under natural conditions. The pellet and/or viscous liquid is thermostated in a water bath at 40° C. and resuspended in 2.0 mL of distilled water (thermostated at 40° C.) under constant stirring. This results in spontaneous development of a clear aqueous solution of 0.4 wt % brinzolamide, latanoprost, brimonidine, and/or bosentan MNF. This aqueous solution is allowed to reach room temperature, under natural conditions. The pH of the solution is adjusted to 5.5 and the volume is made up with 2× phosphate buffer saline (pH 5.5) containing octoxynol-40 (0.01 wt %) and PVP-K-90 (1.2 wt %). The formulation is filtered through 0.2 μm nylon filter to remove any foreign material and obtain a clear homogenous aqueous formulation.

¹H NMR Qualitative Studies:

To determine the absence of free drug in the outer aqueous environment, qualitative studies are conducted. Qualitative proton nuclear magnetic resonance (NMR) studies are conducted with Varian 400 MHz NMR. Deuterated chloroform and water as solvent systems are used to resuspend the formulation and NMR studies were performed.

Results:

Compound added to HCO-40 or HCO-60 at 40° C. can be used to entrap the brinzolamide, latanoprost, brimonidine, and/or bosentan. At higher temperatures the polymer and the drug mixture remain in viscous liquid state. When allowed to reach room temperature, under natural conditions, HCO-60 mixture solidifies and develops a waxy solid. This waxy solid when thermostated at 40° C., helps in resuspending the formulation in distilled water to spontaneously develop brinzolamide and/or bosentan MNF. Similar observations and results are obtained with HCO-40 viscous liquid. The viscosity of the mixture appears to be improved at lower temperatures (4° C.). Therefore, it appears to stick to the walls of the container as thick viscous liquid. Upon allowing return back to room temperature the viscosity appears to be reduced and the mixture retains its flow back.

The waxy solid developed with the mixture of HCO-60 and brinzolamide, latanoprost, brimonidine, and/or bosentan may be helpful to protect the drug and prevent drug degradation with a surface blanket of an inert gas. The other polymer (HCO-40) does not result in development of waxy solid at room temperature or at low refrigerated conditions (4° C.) when used up to approx. 3.0 wt %.

Qualitative proton NMR studies show that resuspending the formulation in the aqueous phase (D₂O) spontaneously generates mixed nanomicelles and no free drug peaks are evident in the aqueous solution. If the drug is not entrapped in the core of mixed nanomicelles then the oil would be floating at the surface as a separate oil phase. While on the otherhand, resuspending the same formulation in organic solvent such as deuterated chloroform (CDCl₃) shows distinct peaks corresponding to drug along with polymer peaks. This indicates that the drug is not encapsulated in the micelle core and is freely available when present in organic solvent.

Conclusions. These studies show that the polymer HCO-60 can be used to entrap brinzolamide, latanoprost, brimonidine, and/or bosentan with Hot Melt method. HCO-40 does not develop into a waxy solid at higher weight percent (3.0 wt %) under the conditions described herein. On the otherhand, HCO-60 develops a waxy solid at 2.0 wt %. This method has unique advantages of being an easy and fast method that avoids the use of organic solvent in the preparation of MNF. Also, the method of preparation is easy and fast. The waxy solid developed in stage 1 may be helpful in preventing drug degradation and help the drug to stay in a waxy solid state at room temperature with a blanket of inert gas. Qualitative proton NMR studies show that drug is not freely available when resuspended in aqueous solution. On the otherhand, when the same formulation is resuspended in organic solvent, CDCl₃, drug peaks are clearly evident indicating the presence of drug in the outer organic solvent environment due to the formation.

### Example 5

### Preparation of Mixed Nanomicellar Brinzolamide, Latanoprost, Brimonidine, and/or Bosentan Formulation

MNF formulation of brinzolamide, latanoprost, brimonidine, and/or bosentan are prepared by solvent evaporation method in two steps: 1. Preparation of basic formulation and 2. rehydration. In step one, brinzolamide, latanoprost, brimonidine, and/or bosentan, HCO-40 and octoxynol-40 are dissolved separately in 0.5 mL ethanol aliquots. These three solutions are mixed together in a round bottom flask. The resultant mixture is stirred to obtain a homogenous solution. Ethanol solvent is removed by high speed vacuum evaporation overnight to obtain a solid thin film.

In step two, the resultant thin film is hydrated with 2.0 mL of double distilled deionized water and resuspended with stirring overnight. The rehydrated formulation is pH adjusted and the volume is made up with 2× phosphate buffer solution, (pH 6.8). Further the formulation is filtered through 0.2 μm nylon filter membrane to remove the unentrapped drug aggregates and other foreign particulates.

Formulations are characterized for their appearance, size and polydispersity indices. The formulations are found to be clear and have very small size with narrow polydispersity index.

Water Method.

MNF formulations of brinzolamide, latanoprost, brimonidine, and/or bosentan can also be prepared by the water method. One mL of double distilled deionized water is heated to 60° C. in a round bottom flask. This heated water is kept under stirring. HCO-40 is added to the heated water and allowed to dissolve under constant stirring. Octoxynol-40 is then added to this mixture and allowed to dissolve. In a separate container, phosphates, sodium chloride and brinzolamide and/or bosentan are blended by hand shaking for a few minutes. Under stirring conditions, the phosphates/brinzolamide/bosentan/sodium chloride blend is added to the solution of HCO-40 and octoxynol-40 to disperse the drug. This mixture is allowed to cool to room temperature while stirring and checked for complete dissolution of drug. PVP K 90 solution is separately prepared using the remaining 1 mL double distilled deionized water. This PVP K 90 solution is added to the solution of polymer/surfactant/drug/phosphate/sodium chloride. Water is added to make up the final volume. Then the formulation is filtered through 0.2 μm nylon membrane to remove the drug aggregates and other foreign particulates.

### Example 6

### Local Tolerability in Rabbits of Formulations

Healthy young adult New Zealand albino rabbits (3-4 Kg) are used for the study of the local tolerability of the instant formulations, for example a formulation of Examples 1-5. One drop (approximately 30. mu.L) of saline is placed in one eye and a drop of formulation is placed in the other eye of the rabbit. Both eyes of each animal are examined by a veterinary ophthalmologist using a hand-held slit lamp and indirect ophthalmoscope. Both control and test eyes are graded according to conjunctival congestion, swelling, and discharge, aqueous flare, iris light reflex and involvement, corneal cloudiness severity and area, pannus, fluorescein examination and lens opacity using the Hackett/McDonald scoring system (see, for example, Hackett, R. B. and McDonald, T. O. Ophthalmic Toxicology and Assessing Ocular Irritation. Dermatoxicology, 5.sup.th Edition. Ed. F. N. Marzulli and H. I. Maibach. Washington, D.C.: Hemisphere Publishing Corporation. 1996; 299-305 and 557-566.).

In the fluorescein examination, approximately one drop of 0.9% sodium chloride, USP, is applied to the end of a fluorescein impregnated strip and then applied to the superior sclera of the left and right eyes (one fluorescein impregnated strip is used for each animal). After an approximate 15 second exposure, the fluorescein dye is gently rinsed from each eye with 0.9% sodium chloride, USP. The eyes are then examined using a slit lamp with a cobalt blue filtered light source. For the lenticular examination approximately one drop of a short-acting mydriatic solution is instilled onto each eye in order to dilate the pupil. After acceptable dilation has occurred, the lens of each eye is examined using a slit-lamp biomicroscope.

The crystalline lens is observed with the aid of the slit-lamp biomicroscope, and the location of lenticular opacity is discerned by direct and retro illumination. The location(s) of lenticular opacities are arbitrarily divided into the following lenticular regions beginning with the anterior capsule:
Anterior subcapsular,
Anterior cortical Nuclear Posterior cortical,
Posterior subcapsular, and
Posterior capsular.
   The lens is evaluated routinely during ocular evaluations and graded as either 0 (normal) or 1 (abnormal). The presence of lenticular opacities are described and the location noted.

### Example 7

### Ocular Tissue Distribution of Formulations of 0.05 wt %, 0.2 wt % and 0.5 wt % in Mixed Micellar Formulations of the Present Disclosure

The temporal distribution and potential accumulation of (ophthalmic solution) of the present disclosure (for example the formulations of Examples 1-5) is assessed after ocular application as a function of repeat dosing, gender difference, and potential melanin binding. This assessment is carried out by determining the concentration of active ingredients in ocular tissues, tears, and blood in New Zealand White (NZW) and Dutch Belted (DB) rabbits.

NZW rabbits are used in single dose (SD) and 7-day repeat dose (RD) studies. DB rabbits are also used in the single dose study. Animals are either untreated (controls) or given a single or a daily topical ocular dose for 7 days (0.05 wt %, 0.2 wt % or 0.5 wt % in a mixed micellar formulation to one or both eyes). Blood and ocular tissue concentrations are assessed.

The concentration of drug is in tissues in the front of the eye (cornea, conjunctiva, sclera) and at the back of the eye (retina, optic nerve) but minimal in the middle of the eye (aqueous and vitreous humor), suggesting transport of the drug by a mechanism other than passive transport through the eye. The high drug levels achieved at the back of the eye make topical administration of the compositions of the present disclosure feasible for the treatment of diseases of the back-of-the-eye (e.g., retinal, diseases involving optic nerve such as glaucoma). Very high levels, especially in target tissues such as lachrymal gland, are achieved with the compositions of the present disclosure.

### Example 8

### Use of Mixed Nanomicellar Formulations for Treating Dry Eye

Mixed nanomicellar formulations according to Examples 1-5 are administered to a patient having dry eye at a concentration of between 0.05% and 0.2% b.i.d. over a period of 1 month to 1 year or more.

### Example 9

### Use of Mixed Nanomicellar Formulations for Treating Diabetic Retinopathy

Mixed nanomicellar formulations according to Examples 1-5 are administered to a patient having proliferative diabetic retinopathy at a concentration of between 0.2 wt % to 0.5 wt % b.i.d. over a period of 1 month to 1 year or more.

### Example 10

### Tolerance and Ocular Tissue Distribution of Brinzolamide, Latanoprost, Brimonidine, and/or Bosentan Mixed Nanomicellar Formulations

A study is conducted in rabbits to test the tolerance and ocular tissue distribution of a nanomicellar formulation of brinzolamide, latanoprost, brimonidine, and bosentan against placebo therefor and balanced saline solution (BSS). Healthy New Zealand female white rabbits (2-3 kg) are used for this study. Brinzolamide, latanoprost, brimonidine, and/or bosentan study drug was prepared having 0.1% brinzolamide, latanoprost, brimonidine, or bosentan essentially as described in the examples herein. The Table below shows the formulation composition of the active formulation and the Placebo.

TABLE
Formulation Composition

| | Active agent 0.1 wt % | |
|---|---|---|
| Components | formulation | Placebo |
| Brinzolamide, latanoprost, | 0.1% | 0 |
| brimonidine, or bosentan | | |
| Hydrogenated castor oil-40 | 1.0% | 1.0% |
| Octoxynol-40 | 0.05% | 0.05% |
| Sodium chloride | 0.10% | 0.10% |
| PVP-K90 | 0.60% | 0.60% |
| Disodium EDTA | 0.05% | 0.05% |
| Benzalkonium chloride | 0.003% | 0.003% |
| Sodium Phosphate buffer | ~0.4% | ~0.4% |
| pH | 7 | 7 |

One drop (approximately 35 μL) of study drug is applied o.d. 4×/day at two hour intervals for 5 days. One drop of BSS is applied to the contralateral eye.

The tolerance parameters evaluated are: physical examination (acclimation study release); viability (daily); clinical observations (daily); Hackett-McDonald Ocular Irritation scores (pre-dose baseline data for each rabbit and then a pre-dose [prior to first daily dose] each day and then 30 min after last dose daily, intraocular pressure (TOP) pre-dose baseline data for each rabbit and then 30 minutes after the evening examinations each day, electroretinography (ERG) pre-dose-(pre-study) baseline data for each rabbit and then one hour after the last treatment, and ocular histopathology at euthanasia.

Mean cumulative Hackett-McDonald ocular irritation scores demonstrate very minimal scores for both BSS-treated left eyes and cyclosporine treated right eyes throughout the study, both for pre-treatment and post-treatment examination times. Mean cumulative inflammatory scores of less than 2 are observed in eyes treated with the therapeutic agent, placebo, and BSS. These clinical scores represented mild conjunctival hyperemia (redness) and swelling. However, there are no significant differences in mean cumulative Hackett-McDonald ocular irritation scores between the groups, suggesting no difference in irritation from topical application of 0.1% therapeutic agent in HCO-40, the HCO-40 placebo, and BSS.

No changes in TOP are noted in eyes treated with BSS, HCO-40, or therapeutic agent. No toxicologic changes in retinal function are noted on ERG after 5 days of treatment with the test articles. No toxicologic or inflammatory changes are observed histologically in the anterior (conjunctiva/cornea/iris) or posterior segments (vitreous/retina) of the eye of any groups.

Samples of selected ocular tissues (aqueous humor, vitreous humor, conjunctiva, cornea, iris-ciliary body, lens, retina/choroid, and sclera) are collected 1 hour following the last dose on Day 5 from all rabbits that receive 0.1% therapeutic agent with HCO-40 (OD), and BSS (OS), and from one rabbit (No. 21) that received placebo HCO-40 formulation (OD) and BSS (OS). The samples are assayed for therapeutic agent by liquid chromatography-tandem mass spectrometry (LC-MS/MS). The internal standard is d₄-brinzolamide, d₄-latanoprost, d₄-brimonidine, or d₄-bosentan. The established analytical ranges for the therapeutic agent are 0.100-100 ng/mL for whole blood, and 2.00-2000 ng/mL for aqueous humor and vitreous humor. The analytical ranges for the solid tissues are 0.125-30 ng (low range) and 1.00-2500 ng (high range). The results of the solid tissue analyses are converted to ng/g by correcting for the amount of tissue analyzed.

Following repeated administration of the 0.1% therapeutic agent-HCO-40 formulation, the highest average therapeutic agent concentrations in the treated eye are observed in the cornea, followed by conjunctiva, sclera, iris-ciliary body, and aqueous humor. The lowest therapeutic agent concentrations are observed in the lens, retina/choroid, and vitreous humor. Concentrations of the therapeutic agent in the collateral eye treated with BSS are quite low suggesting minimal systemic transfer of drug.

The ocular tissue concentrations for the 0.1% therapeutic agent formulation observed herein are generally higher than the Cₘₐₓ values following repeat dose administration (bid for 7 days) of an Allergan 0.2% 3H cyclosporine A formulation to rabbits (see Acheampong A A, Shackleton M, Tang-Liu D, Ding S, Stern M E, Decker R Distribution of cyclosporin A in ocular tissues after topical administration to albino rabbits and beagle dogs; Current Eye Research 18(2); 1999; pp 91-103).

### Example 11

### Tolerance and Ocular Tissue Distribution of Therapeutic Agent Mixed Nanomicellar Formulations

A study is conducted in rabbits to test the tolerance and ocular tissue distribution of two nanomicellar formulations of therapeutic agent against matching placebos and balanced saline solution (BSS). Healthy New Zealand female white rabbits (2-3 kg) are used for this study. One drop (approximately 35 μL) of study drug is applied o.d. 4×/day at two hour intervals for 5 days. One drop of BSS is applied to the contralateral eye.

The tolerance parameters evaluated are: physical examination (acclimation study release); viability (daily); clinical observations (daily); Hackett-McDonald Ocular Irritation scores (pre-dose baseline data for each rabbit and then a pre-dose [prior to first daily dose] each day and then 30 min after last dose daily, intraocular pressure (TOP) pre-dose baseline data for each rabbit and then 30 minutes after the evening examinations each day, electroretinography (ERG) pre-dose-(pre-study) baseline data for each rabbit and then one hour after the last treatment, and ocular histopathology at euthanasia.

Cumulative Hackett-McDonald ocular irritation scores demonstrate very minimal mean values for both BSS-treated left eyes and test-article treated right eyes throughout the study, both for pre-treatment and post-treatment examination times. There are no significant differences in mean cumulative Hackett-McDonald ocular irritation scores between the groups. The observed ocular irritation is interpreted as minimal and transient in all groups.

No changes in TOP are noted in eyes treated with BSS or test articles. No toxicologic changes in retinal function are noted on ERG after 5 days of treatment with the test articles. No toxicologic or inflammatory changes are observed histologically in the anterior (conjunctiva/cornea/iris) or posterior segments (vitreous/retina) of the eye of any groups.

Selected ocular fluids/tissues (aqueous humor, vitreous humor, conjunctiva, cornea, iris-ciliary body, lens, retina/choroid, and sclera) collected from two rabbits each in the therapeutic agent (0.15 wt % in HCO-60, 0.1 wt % in HCO-40) treatment groups, and from one rabbit in each of the matching placebo groups, are assayed for therapeutic agent by liquid chromatography-tandem mass spectrometry (LC-MS/MS). Warfarin-d₅ and 5-HDA are used as internal standards for the analysis of therapeutic agent in aqueous humor and vitreous humor. For the other ocular tissues (solid tissues), warfarin-d5 and phenyl acetic acid-d5 (PAA-d₅) are used as the internal standards for brinzolamide and bosentan, respectively. The analytical range for the solid tissues is 0.125-100 ng. The results of the solid tissue analyses are converted to ng/g by correcting for the amount of tissue analyzed.

Only sporadic, relatively low, concentrations of therapeutic agent are observed in the sclera and conjunctiva. Therapeutic agent is either not detected or is below the quantitation limit of the assay in the majority of ocular tissues.

The invention illustratively described herein may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other documents.

The inventions illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

Other embodiments are set forth within the following claims.

What is claimed is:

1. An ophthalmic formulation comprising:
at least one active agent selected from the group consisting of brinzolamide, latanoprost, brimonidine, and bosentan,
a polyoxyl lipid or fatty acid and
a polyalkoxylated alcohol.

2. The ophthalmic formulation of claim 1, wherein said polyoxyl lipid comprises one or more selected from the group consisting of HCO-40, HCO-60, HCO-80, HCO-100, polyoxyl 40 stearate and polyoxyl 35 castor oil.

3. The ophthalmic formulation of claim 1, wherein said polyoxyl lipid comprises one or more selected from the group consisting of HCO-40, HCO-60 and HCO-80.

4. The ophthalmic formulation of claim 1, wherein said polyoxyl lipid comprises HCO-40.

5. The ophthalmic formulation of claim 1, wherein said polyoxyl lipid comprises one or more selected from the group consisting of HCO-60, HCO-80, HCO-100, polyoxyl 40 stearate and polyoxyl 35 castor oil and is present in an amount between 0.05-5 wt % of the formulation.

6. The ophthalmic formulation of claim 1, wherein said polyoxyl lipid comprises one or more selected from the group consisting of HCO-40, HCO-60, HCO-80, HCO-100, polyoxyl 40 stearate and polyoxyl 35 castor oil and is present in an amount between 0.5-1.5 wt % of the formulation.

7. The ophthalmic formulation of claim 1, wherein said polyoxyl lipid comprises one or more selected from the group consisting of HCO-40, HCO-60, and HCO-80, and is present in an amount between 0.5-1.5 wt % of the formulation.

8. The ophthalmic formulation of claim 1, wherein said polyalkoxylated alcohol is Octoxynol-40.

9. The ophthalmic formulation of claim 1, wherein the polyalkoxylated alcohol is Octoxynol-40 and is present in an amount between 0.02 and 4 wt % of the formulation.

10. The ophthalmic formulation of claim 1, wherein the polyalkoxylated alcohol is Octoxynol-40 and is present in an amount between 0.02 and 0.1 wt % of the formulation.

11. The ophthalmic formulation of claim 1, wherein the active agent is present in an amount between 0.05 and 5 wt % of the formulation.

12. The ophthalmic formulation of claim 1, wherein the active agent is present in an amount between 0.05 and 0.2 wt % of the formulation.

13. The ophthalmic formulation of claim 1, wherein said polyoxyl lipid comprises one or more selected from the group consisting of HCO-40, HCO-60, HCO-80, HCO-100, polyoxyl 40 stearate and polyoxyl 35 castor oil; and wherein said said polyalkoxylated alcohol is Octoxynol-40.

14. The ophthalmic formulation of claim 1, wherein said polyoxyl lipid comprises one or more selected from the group consisting of HCO-40, HCO-60, HCO-80, HCO-100, polyoxyl 40 stearate and polyoxyl 35 castor oil and is present in an amount between 0.5-5 wt % of the formulation; and said polyalkoxylated alcohol is Octoxynol-40 and is present in an amount between 0.02 and 4 wt % of the formulation.

15. The ophthalmic formulation of claim 1, wherein said polyoxyl lipid comprises one or more selected from the group consisting of HCO-40, HCO-60, HCO-80, HCO-100, polyoxyl 40 stearate and polyoxyl 35 castor oil and is present in an amount between 0.5-1.5 wt % of the formulation; and said polyalkoxylated alcohol is Octoxynol-40 and is present in an amount between 0.02 and 0.1 wt % of the formulation.

16. The ophthalmic formulation of claim 1, wherein said polyoxyl lipid comprises one or more selected from the group consisting of HCO-40, HCO-60, HCO-80, HCO-100, polyoxyl 40 stearate and polyoxyl 35 castor oil and is present in an amount between 0.5-5 wt % of the formulation; said polyalkoxylated alcohol is Octoxynol-40 and is present in an amount between 0.02 and 4 wt % of the formulation; and the active agent is present in an amount between 0.05 and 5 wt % of the formulation.

17. The ophthalmic formulation of claim 1, wherein said polyoxyl lipid comprises one or more selected from the group consisting of HCO-40, HCO-60, HCO-80, HCO-100, polyoxyl 40 stearate and polyoxyl 35 castor oil and is present in an amount between 0.5-1.5 wt % of the formulation; said polyalkoxylated alcohol is Octoxynol-40 and is present in an amount between 0.02 and 0.1 wt % of the formulation; and the active agent is present in an amount between 0.05 and 0.2 wt % of the formulation.

18. The ophthalmic formulation of claim 1, wherein said formulation comprises nanomicelles.

19. The ophthalmic formulation of claim 1, wherein said active agent is brinzolamide.

20. The ophthalmic formulation of claim 1, wherein said active agent is bosentan.

21. The ophthalmic formulation of claim 1, wherein said active agent is latanoprost.

22. The ophthalmic formulation of claim 1, wherein said active agent is brimonidine.

23. A method of treating or preventing an ocular disease or condition, said method comprising topically administering a formulation of claim 1.

24. A method of treating or preventing an ocular disease or condition, said method comprising topically administering a formulation of claim 17.
